(19) United States
(12) Patent Application Publication
(10) Pub. No.: US 2021/0244340 A1
(43) Pub. Date: Aug. 12, 2021

(54) METHOD OF PROVIDING VENTRICULAR ARRHYTHMIA LOCALIZATION AND MYOCARDIUM WALL THICKNESS WITHIN A 3D HEART MODEL

(71) Applicant: CATHETER PRECISION, INC., Mt. Olive, NJ (US)

(72) Inventor: Barry Yomtov, Marblehead, MA (US)

(21) Appl. No.: 17/174,308

(22) Filed: Feb. 11, 2021

Related U.S. Application Data
(60) Provisional application No. 62/975,118, filed on Feb. 11, 2020.

(51) Int. Cl. A61B 5/363; A61B 5/00; A61B 5/055; A61B 5/28

(52) U.S. Cl. CPC A61B 5/363; A61B 5/742; A61B 5/6823; A61B 5/28; A61B 5/055

(57) ABSTRACT

Various embodiments include methods and computing systems for arrhythmia localization and display. A computing system may include generating a patient-specific three-dimensional (3D) heart model including a 3D internal surface model, such as based on medical image data, generating a patient-specific electrical conduction model of a patient's heart of an arrhythmia based on the patient-specific 3D heart model and electrocardiogram (ECG) data. The patient-specific electrical conduction model of the patient's heart may identify a localization of an initiation site of the arrhythmia The computing system may merge the 3D localization of the initiation site of the arrhythmia and the 3D internal surface model to form an arrhythmia activation surface model, and generate a 3D model of the heart showing the wall thickness of the heart's myocardium simultaneously with the localization of an arrhythmia.

### CROSS-REFERENCE TO RELATED APPLICATION

This application claims the benefit of priority to U.S. Provisional Patent Application No. 62/975,118, entitled “Method of Providing Ventricular Arrhythmia Localization and Myocardium Wall Thickness Within a 3D Heart Model”, filed Feb. 11, 2020, the entire contents of which are herby incorporated by reference for all purposes.

### BACKGROUND

Some heart defects in the conduction system result in asynchronous contraction (arrhythmia) of the heart and are sometimes referred to as conduction disorders. As a result, the heart does not pump enough blood, which may ultimately lead to heart failure. Conduction disorders can have a variety of causes, including age, heart (muscle) damage, medications and genetics.

Premature Ventricular Contractions (PVCs) are abnormal or aberrant heart beats that start somewhere in the heart ventricles rather than in the upper chambers of the heart as with normal sinus beats. PVCs typically result in a lower cardiac output as the ventricles contract before they have had a chance to completely fill with blood. PVCs may also trigger Ventricular Tachycardia (VT or V-Tach).

Ventricular tachycardia (VT or V-Tach) is another heart arrhythmia disorder caused by abnormal electrical signals in the heart ventricles. In VT, the abnormal electrical signals cause the heart to beat faster than normal, usually more than 100 beats per minute, with the beats starting in the heart ventricles. VT generally occurs in people with underlying heart abnormalities. VT can sometimes occur in structurally normal hearts, and in such patients the origin of abnormal electrical signals can be in multiple locations in the heart. One common location is in the right ventricular outflow tract (RVOT), which is the route the blood flows from the right ventricle to the lungs. In patients who have had a heart attack, scarring from the heart attack can create a milieu of intact heart muscle and a scar that predisposes patients to VT.

### SUMMARY

Various embodiments include methods and computing systems implementing the methods for arrhythmia localization and display for use in a medical procedure. Various embodiments may include generating a patient-specific three-dimensional (3D) heart model including a 3D internal surface model, generating a patient-specific electrical conduction map of a patient's heart of an arrhythmia based on the patient-specific 3D heart model and electrocardiogram (ECG) data, the patient-specific electrical conduction map of the patient's heart identifying a localization of an initiation site of the arrhythmia, merging the 3D localization of the initiation site of the arrhythmia and the 3D internal surface model to form an arrhythmia activation surface model, generating a 3D model of the heart showing the wall thickness of the heart's myocardium simultaneously with the localization of an arrhythmia, and displaying the 3D localization of the initiation site of the arrhythmia simultaneously with heart wall thickness of the 3D heart model of the myocardium for use by a physician during a cardiac electrophysiology procedure. Some aspects may further include supplementing the patient-specific electrical conduction map by an internal point by point contact recording.

In some embodiments, generating a patient-specific 3D model of the heart including a 3D internal surface model may include using magnetic resonance imaging (MRI) or computed tomography (CT) images of the patient to generate the patient-specific 3D heart model. Some embodiments may further include, obtaining a 3D image of ECG electrodes on the patient's torso, and merging the 3D image of the patient's torso with the 3D heart model. In some embodiments, merging the 3D image of the patient's torso with the 3D patient specific heart model may include aligning locations of ECG electrodes used in generating patient-specific electrical conduction map of a patient's heart with the ECG electrodes within the 3D image. In some embodiments, ECG data obtained with 12 ECG electrodes is combined with the patient specific 3D heart model using an inverse solution calculation to generate a localization the initiation site of the arrhythmia in a heartbeat. Some embodiments may further include superimposing the localization point on the 3D heart model that includes the myocardium wall thickness. Some embodiments may further include displaying heart structures including one or more of the aorta, aortic arch, pulmonary veins or coronary vessels on the displayed 3D heart model. Some embodiments may further include displaying heart scar tissue indicative of ischemic heart disease on the displayed 3D heart model. Some embodiments may further include displaying the localization of the arrhythmia as multiple points representative of multiple beats of a ventricular tachycardia on the displayed 3D heart model.

Further embodiments include a computing system having a memory storing a database of representative 3D heart models, and a processor coupled to the memory and configured with processor-executable instructions to perform operations of any of the embodiments summarized above.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a system block diagram of a computing system configured to perform operations of various embodiments.

FIGS. 2A and 2B are illustrations of a typical human heart.

FIG. 3 is an example of a 3D heart model of the patient specific heart model providing the heart wall thickness that may be stored within the heart model data base according to various embodiments.

FIG. 4 illustrates example of a 3D heart model with results of a localization and cardiac activation map of a single arrhythmia event.

FIG. 5 illustrates a 3D heart model with results of a localization of a single arrhythmia event, superimposed on the heart wall thickness.

FIG. 6 illustrates an example operational workflow for an embodiment method for generating a localization of an arrhythmia according to the second embodiment.

FIGS. 7A-7C are process flow diagrams illustrating methods for generating arrhythmia activation surface models suitable for use in a medical procedure according to some embodiments.

FIG. 8 is a component block diagram illustrating an example mobile computing device suitable for use with the various embodiments.

FIG. 9 is a component block diagram illustrating an example server suitable for use with the various embodiments.

### DETAILED DESCRIPTION

The various embodiments will be described in detail with reference to the accompanying drawings. References made to particular examples and implementations are for illustrative purposes, and are not intended to limit the scope of the invention or the claims.

Catheter ablation is a treatment of choice in patients with VT and/or symptomatic PVCs. The targets for ablation are locations in the heart where PVCs are occurring or locations where the onset of the VT is occurring. In order to determine a proper ablation location, a treating physician may first stimulate a proposed location using an electrical lead, in order to determine whether ablation at the proposed location will provide a desired electrical activation pattern stimulation of the heart. At times, there is a need to determine where the area of earliest activation (or localization) of the PVC within the heart's myocardium. By also providing the wall thickness information, this will allow the physician to set the proper ablation parameters, for example energy, temperature, etc. This process will provide for a safer result during a transmural lesion.

Various embodiments include methods of identifying and displaying the location in the heart of the earliest activation of an arrythmia, which is referred to herein as “localization” of an initiation site of the arrhythmia Various embodiment methods may include generating a patient-specific 3D heart model, such as based on medical imaging information (e.g., MRI and/or CT scans). A patient-specific electrical conduction map of an arrhythmia in a patient's heart may be developed based on the patient-specific 3D heart model and ECG data. The patient-specific electrical conduction map of the patient's heart may identify the location or localization of an initiation site of the arrhythmia The 3D localization of the initiation site of the arrhythmia and the 3D internal surface model may be merged to form an arrhythmia activation surface model. A 3D model of the heart may be generated showing the wall thickness of the heart's myocardium simultaneously with the localization of an arrhythmia The 3D localization of the initiation site of the arrhythmia may be displayed (or output for display) simultaneously with heart wall thickness of the 3D heart model of the myocardium for use in a medical procedure, such as an electrophysiology therapy (e.g., ablation therapies).

An electrocardiogram (ECG) is defined herein as any method that (preferably non-invasively) correlates actual electrical activity of the heart muscle to measured or derived (electrical activity) of the heart. In case of a classical electrocardiogram, the differences in potential between electrodes on the body surface are correlated to the electrical activity of the heart. In order to obtain such a functional image an estimation of the movement of the electrical activity has to be provided.

As used herein, an electrocardiogram (ECG) is any method that (preferably non-invasively) correlates actual electrical activity of the heart muscle to measured or derived (electrical activity) of the heart. In case of a classical electrocardiogram, the differences in potential between electrodes on the body surface are correlated to the electrical activity of the heart. In order to obtain a functional image, an estimation of the movement of the electrical activity may be provided. As used herein, the term “electrocardiographic data” refers to graphical information related to or displaying ECG data.

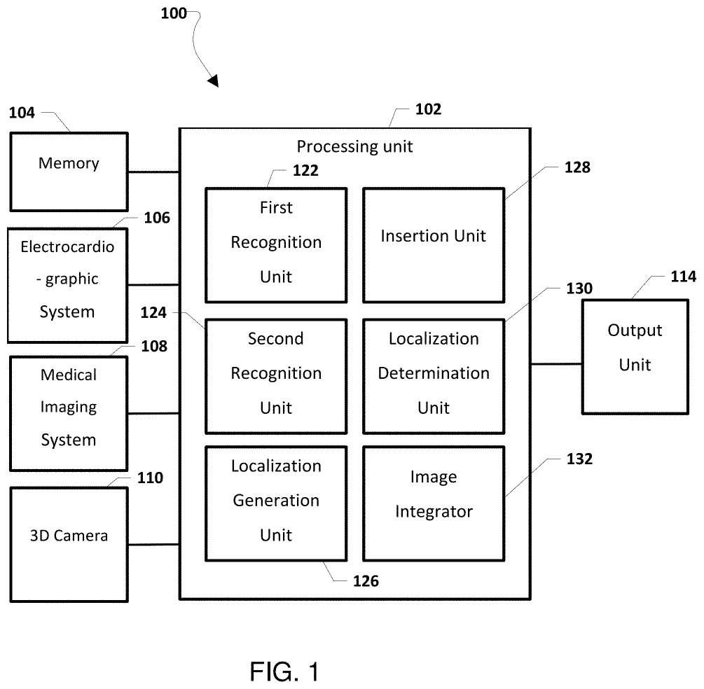

FIG. 1 is a block diagram providing a schematic representation of a system for providing a representation of arrythmia initialization localization within the heart tissue. The computing system 100 may include a processing unit 102, a memory 104, an electrocardiographic system 106, a 3D camera 110, and an output unit 114, such as a display or a network connection for outputting display dated to a remote display unit (not shown). The computing system 100 may receive input data in the form of electrocardiographic (ECG) data generated by an ECG system 106, a picture of the patient's torso with ECG electrodes attached captured by a 3D camera 110, and medical image data captured by a medical imaging system 108, such as a magnetic resonance imaging (MRI) and/or a computed tomorgraphy (CT) system. Some or all of such input data may be obtained from memory, including memory accessed via a network, memory of an attached device, or portable memory coupled to the computing system, or received directly from an attached device (e.g., the ECG system or 3D camera).

The processing unit 102 may include a first recognition unit 122, a second recognition unit 124, a localization generation unit 126, and insertion unit 128, a localization determination unit 130, and an image integrator 132. These units may be separate processing systems, software modules of processor-executable instructions configured to be executed in a single processor within the processing unit, dedicated hardware, or combinations thereof

The 3D localization of the initiation site of an arrythmia (e.g., a PVC) within the heart tissue may be obtained by the processing unit 102 combining electrocardiographic (ECG) and medical imaging data (MRI or CT). This data may be stored in the memory 104. The processing unit 102 may be connected to an electrocardiographic system 106, a medical imaging system 108, and a 3D camera 110, for retrieving the data and storing corresponding data in the memory 104.

An electrocardiographic imaging (ECGI) method able to determine the localization of the ECG within the heart tissue from a 12 lead ECG may be applied by the processing unit 102 for determining the localization of arrhythmias. The ECG signals may be combined by the processing unit 102 with a patient-specific 3D anatomical model of the heart, lungs, and/or torso (referred to herein as , in order to compute the positions of cardiac isochrones. The patient-specific 3D anatomical heart model may be obtained or generated by the processing unit 102 from MRI or CT images obtained from the medical imaging system 108.

Alternatively or additionally, a 3D anatomical model showing closest conformity to the patient may be selected, and optionally modified, from a database including a plurality of 3D anatomical models. The selected, and optionally modified, 3D anatomical model may serve as the patient-specific 3D anatomical model. Optionally, the patient-specific 3D model may also include the size, orientation and/or location of other structures in the patient, such as the lungs and/or other organs as well as cardiac blood vessels and/or venison the myocardium surface. The patient-specific 3D model may be a volume conductor model. Such a model would provide detailed structures of the heart as shown in the following two figures.

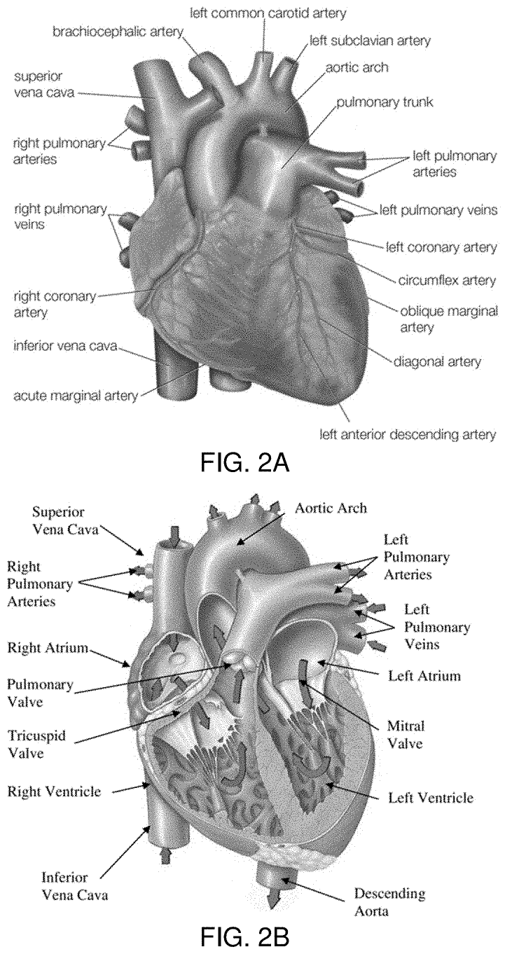

The patient-specific 3D anatomical he art model may include anatomical details such as the aorta, aortic arch, and pulmonary veins. FIGS. 2A and 2B illustrate parts of a typical human heart for reference. For patients who also have ischemic heart disease, the patient-specific 3D anatomical heart model may include or identify regions of scar tissue in relation to the arrythmia localization, heart structures and the heart wall thickness. Including such details in the patient-specific 3D anatomical he art model may help a physician performing an electrophysiology procedures, such as by providing an accurate map for use in guiding a catheter for delivering therapy to heart tissue, such as an RF ablation.

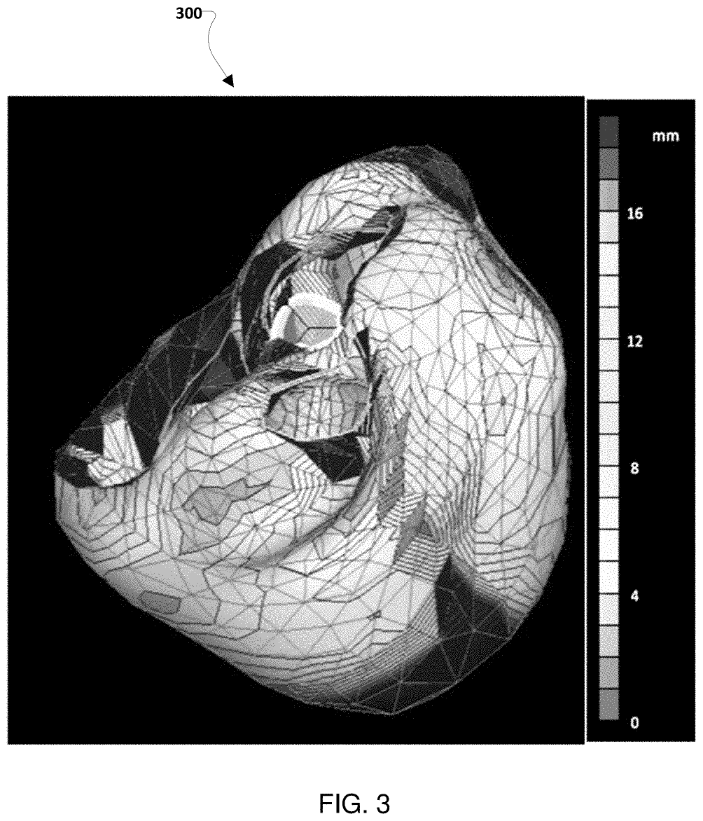

FIG. 3 an example of a patient-specific 3D anatomical he art model that may be generated based on MRI and/or CT images. In FIG. 2, the scale represents the thickness of the heart wall (i.e., the myocardium) in mm. The lighter shades, which could be displayed in light color, represent thinner myocardium and the darker shades, which could be displayed in darker (e.g., green to blue) colors, represent the thicker myocardium.

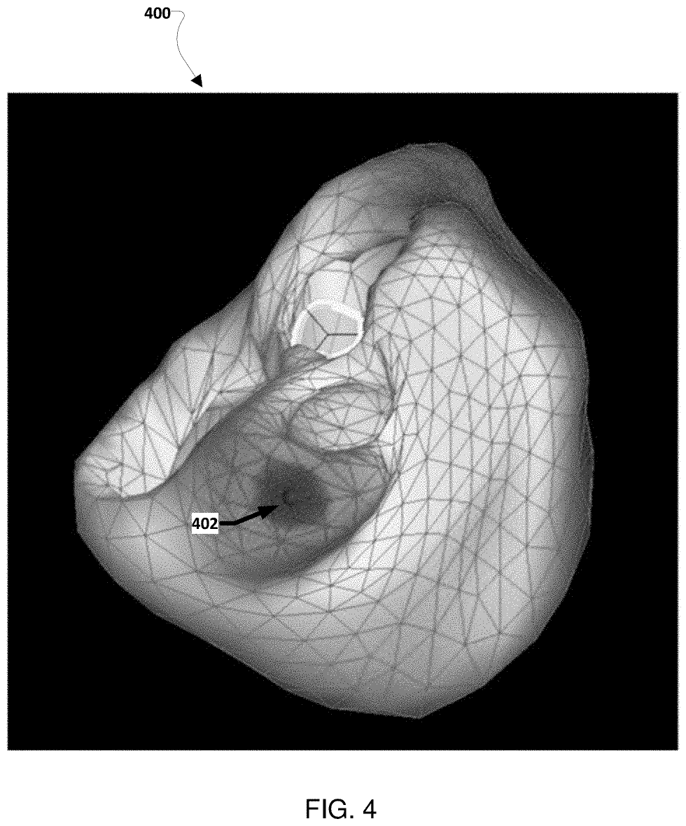

FIG. 4 illustrates example of a 3D heart model 400 with results of a localization and cardiac activation map of a single arrhythmia event, such as a PVC event, that may be produced by a computing system 100 implementing various embodiment methods. In this example, the localization point is on the epicardial surface of the myocardium displayed as a dot 402. The gradient displays the electrical activation of a PVC heartbeat during the QRS complex.

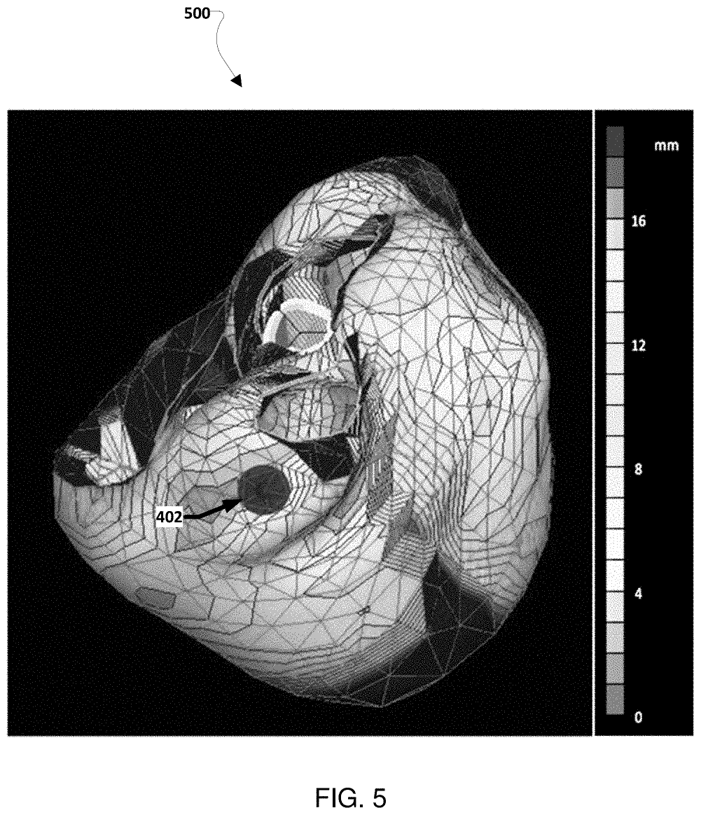

FIG. 5 illustrates an example of the results of superimposing the localization point 402 on the epicardial surface of the patient-specific 3D anatomical heart model 500 while simultaneously displaying the wall thickness of the heart's myocardium. To assist in guiding an ablation catheter to the optimal therapy location, superimposing the localization point 402 on the patient specific 3D heart model provides a more accurate estimate of where and how far to insert the ablation catheter within the myocardium. Displaying the localization point simultaneously with the wall thickness provides the physician additional information for setting the proper ablation parameters for example, ablation energy and temperature for safer transmural lesion results.

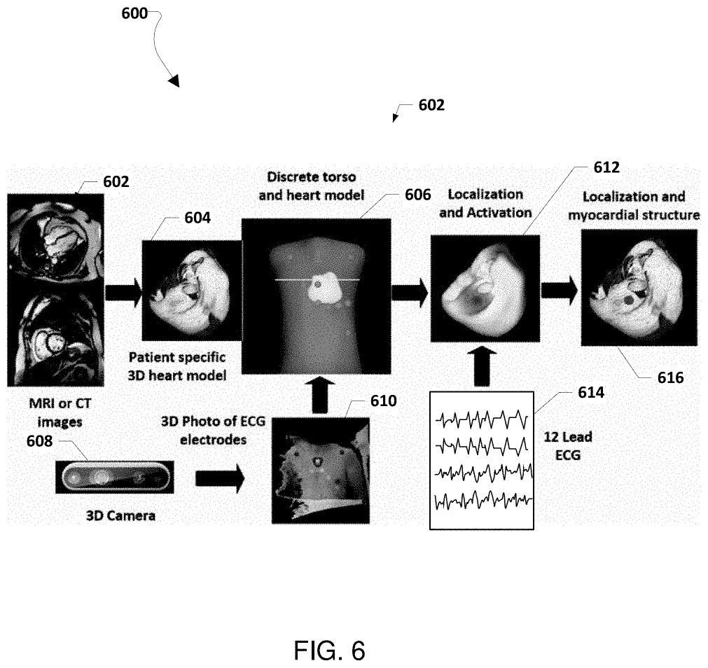

FIG. 6 illustrates an example operational workflow 600 for generating the localization of an arrythmia initiation site, such as localization of a PVC, and superimposing the localization on a patient-specific 3D heart model including the myocardium wall thickness.

MRI or CT image data 602 may be combined and aligned with a selected heart model from a database as a patient specific 3D heart model 604. This heart model 604 may be inclusive of calculation of the inverse solution for the localization of a PVC from an ECG. A patient specific torso model may also be created from the MRI or CT scan data and inclusive of the heart model.

A 3D camera 608 may be used to take a 3D image 610 may be taken of the patient's torso inclusive of ECG electrodes used for a 12-lead electrocardiograph recording 614. The 3D image 610 may be merged with the patient specific 3D heart and torso model 606, with the ECG electrodes aligned between the 3D image 610 and electrode positions in the torso model 606. ECG markers may be placed in certain locations within and arrhythmia heartbeat, such as within the QRS waveform of a PVC. A mathematical model may be used for generating the inverse solution from the 12 lead ECG recording 614 within the timing of the ECG markers to calculate the localization and cardiac activation map of arrhythmias (e.g., a PVC) 612 and generating a localization and myocardial structure map 616.

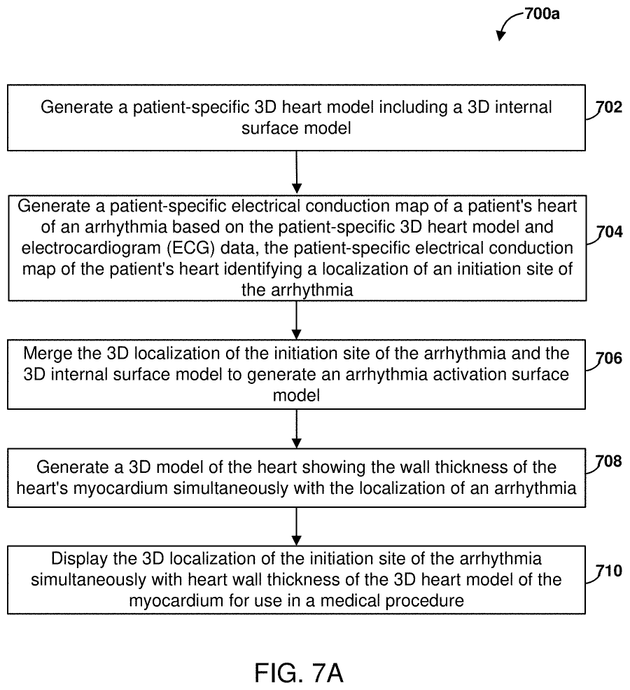

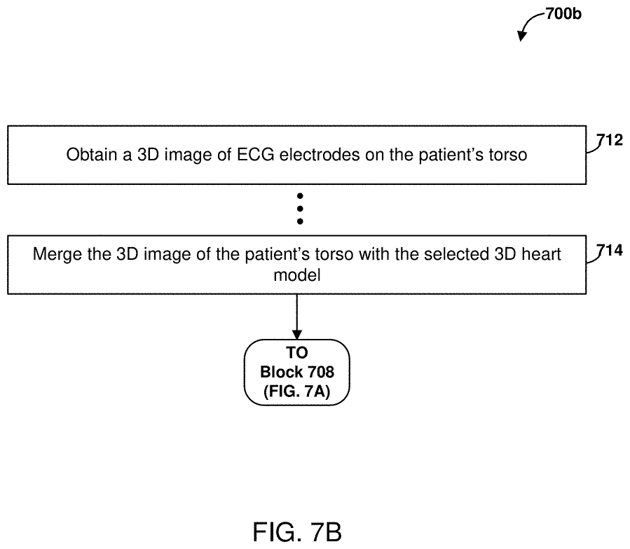

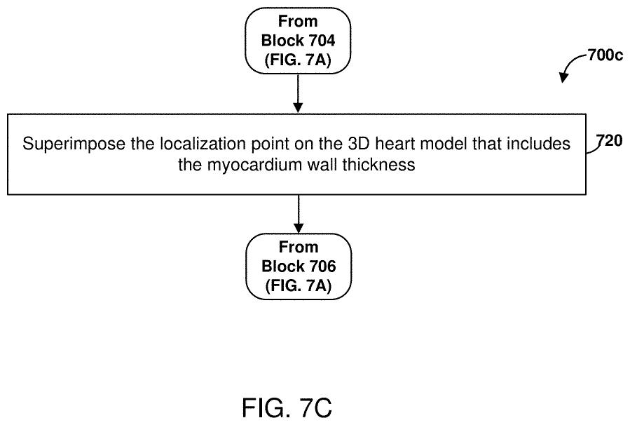

FIG. 7A is a process flow diagram illustrating an embodiment method 700a for determining a likelihood of success and/or complications from performing an ablation procedure to cure arrythmia at a particular location on the heart. The operations of the method 700a may be performed by one or more processors within a processing unit (e.g., 102) of a computing system (e.g., 100).

In block 702, the processing unit 102 may generate a patient-specific 3D heart model including a 3D internal surface model. In some embodiments, the processing unit 102 may generate a patient-specific 3D heart model based on medical imaging system data. In some embodiments, the processing unit 102 may generate a patient-specific 3D heart model using MRI and/or CT images of the patient. In some embodiments, the processing unit 102 may use such image data to measure the myocardium wall thicknesses in various parts of the heart and include the myocardium wall thicknesses in the patient-specific 3D heart model. In some embodiments, the processing unit 102 may use such image data to identify locations and shapes of heart structures, and include one or more of the aorta, aortic arch, pulmonary veins or coronary vessels on the patient-specific 3D heart model. In some embodiments, the processing unit 102 may use such image data to identify locations and shapes of scar tissue in the heart, which may be derived from delayed enhancement MRI images. By including heart structures and/or scar tissue in the patient-specific 3D heart model enables the processing unit 102 to display some or all of such structures.

In block 704, the processing unit 102 may generate a patient-specific electrical conduction map of a patient's heart of an arrhythmia based on the patient-specific 3D heart model and ECG data. The patient-specific electrical conduction map of the patient's heart identifies a localization of an initiation site of the arrhythmia In some embodiments, the processing unit 102 may generate a cardiac activation map as part of the operations in block 704.

In block 706, the processing unit 102 may merge the 3D localization of the initiation site of the arrhythmia and the 3D internal surface model to form an arrhythmia activation surface model. In some embodiments, the processing unit 102 may merge the 3D localization of the initiation site of the arrhythmia and the 3D internal surface model by aligning locations of ECG electrodes used in generating patient-specific electrical conduction map of a patient's heart with the ECG electrodes within a 3D image of the patient's torso.

In block 708, the processing unit 102 may generate a 3D model of the heart showing the wall thickness of the heart's myocardium simultaneously with the localization of an arrhythmia

In block 710, the processing unit 102 may display the patient-specific 3D localization of the arrythmia and the patient-specific cardiac activation map for use in a medical procedure. For example, the processing unit 102 may display the 3D localization of the initiation site of the arrhythmia simultaneously with heart wall thickness of the 3D heart model of the myocardium for use by a physician during a cardiac electrophysiology procedure. In some embodiments, the processing unit 102 may also display the cardiac activation map as part of block 710. In some embodiments, the processing unit 102 may also display the cardiac activation map, either separately or in conjunction with the display of the In some embodiments, localization may be presented with the 3D heart model simultaneously including the heart wall thickness of the myocardium which may aid a physician in the guidance of delivering an electrophysiology therapy, such as a radio frequency ablation therapy. Additional features such as the aorta, aortic arch, pulmonary veins may be added to also aid in the guidance of delivering ablation therapy, for example radio frequency (RF) ablation. Optionally, scar tissue may also be incorporated in the anatomical 3D representation of the heart.

The method 700a may be useful to physicians diagnosing and treating a variety of arrythmia conditions. For example, the processing unit 102 performing the method 700a may be useful for arrythmia localization of atrial arrythmia and ventricular arrhythmia, such as PVC or ventricular tachycardia.

FIG. 7B is a process flow diagram illustrating an example of operations of a method 700b that may be performed to generate a patient-specific 3D heart model useful in merging the 3D localization of the initiation site of the arrhythmia and the 3D internal surface model to generate an arrhythmia activation surface model in block 706 of the method 700a (FIG. 7A). The operations of the method 700b may be performed by one or more processors within a processing unit (e.g., 102) of a computing system (e.g., 100).

In block 712, the processing unit 102 may obtain a 3D image of the ECG electrodes on the patient's torso. This 3D image provides the processing unit with information regarding the orientation and dimensions of the patient's torso as well as the specific locations of each ECG electrode that collect the ECG data used in operations of block 702. The processing unit 102 may obtain the 3D image of the patient's torso during recording of ECG data, but the 3D image may also be taken after the ECG electrodes are attached but before the ECG data is recorded, or after the ECG data has been recorded.

In block 714, the processing unit 102 may merge the 3D image of the patient's torso with the representative 3D heart model selected from the 3D heart model database. In some embodiments, this operation of merging the 3D image of the patient's torso with the 3D patient specific heart model may include aligning the locations of ECG electrodes that are included in the selected 3D heart model with the ECG electrodes within the 3D image. In this manner, the processing unit 102 may generate a patient-specific 3D heart model using the selected representative 3D heart model as a starting point. In some embodiments, ECG data obtained with 12 ECG electrodes is combined with the patient specific 3D heart model using an inverse solution calculation to generate a localization the initiation site of the arrhythmia in a heartbeat.

FIG. 7C is a process flow diagram illustrating an example of operations of a method 700c that may be performed to create or update/adjust representative 3D heart models based on patient demographic data. The operations of the method 700c may be performed by one or more processors within a processing unit (e.g., 102) of a computing system (e.g., 100).

After generating a patient-specific electrical conduction map of a patient's heart of an arrhythmia based on the patient-specific 3D heart model and ECG data, the processing unit 102 may superimpose the localization point on the 3D heart model that includes the myocardium wall thickness in block 720. The processing unit 102 may then perform the operations of block 706 as described.

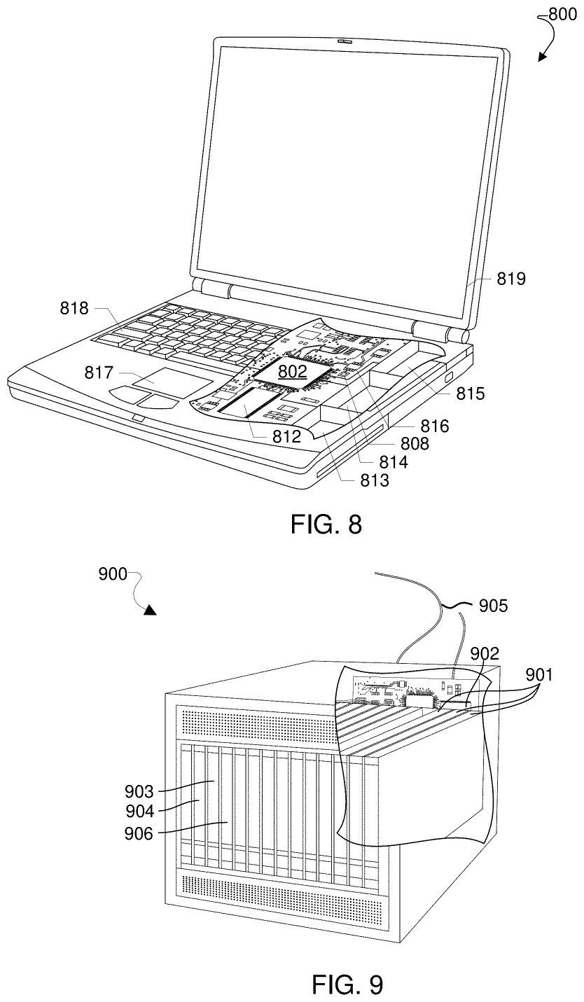

The various embodiments (including, but not limited to, embodiments described above with reference to FIGS. 1-7) may be implemented in a wide variety of computing systems include a laptop computer 800, an example of which is illustrated in FIG. 8. Many laptop computers include a touchpad touch surface 817 that serves as the computer's pointing device. A laptop computer 800 will typically include a processor 802 coupled to volatile memory 812 and a large capacity nonvolatile memory, such as a disk drive 813 or FLASH memory. Additionally, the computer 800 may have one or more antenna 808 for sending and receiving electromagnetic radiation that may be connected to a wireless data link (e.g., Bluetooth or Wi-Fi) and/or cellular telephone transceiver 816 coupled to the processor 802. The computer 800 may also include a floppy disc drive 814 and a compact disc (CD) drive 815 coupled to the processor 802. In a notebook configuration, the computer housing includes the touchpad 817, the keyboard 818, and the display 819 all coupled to the processor 802. Other configurations of the computing device may include a computer mouse or trackball coupled to the processor (e.g., via a USB input) as are well known, which may also be used in conjunction with the various embodiments.

The various embodiments (including, but not limited to, embodiments described above with reference to FIGS. 1-7) may also be implemented in fixed computing systems, such as any of a variety of commercially available servers. An example server 900 is illustrated in FIG. 9. Such a server 900 typically includes one or more multicore processor assemblies 901 coupled to volatile memory 902 and a large capacity nonvolatile memory, such as a disk drive 904. As illustrated in FIG. 9, multicore processor assemblies 901 may be added to the server 900 by inserting them into the racks of the assembly. The server 900 may also include a floppy disc drive, compact disc (CD) or digital versatile disc (DVD) disc drive 906 coupled to the processor 901. The server 900 may also include network access ports 903 coupled to the multicore processor assemblies 901 for establishing network interface connections with a network 905, such as a local area network coupled to other broadcast system computers and servers, the Internet, the public switched telephone network, and/or a cellular data network.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiment methods may be performed in any order.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof If implemented in software, the functions may be stored as one or more instructions or code on a non-transitory computer-readable medium or non-transitory processor-readable medium. The steps of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the claims. Thus, the present invention is not intended to be limited to the embodiments shown herein but is to be accorded the widest scope consistent with the following claims and the principles and novel features disclosed herein.

What is claimed is:

1. A method of displaying arrhythmia localization, comprising:
generating a patient-specific three-dimensional (3D) heart model including a 3D internal surface model;
generating a patient-specific electrical conduction map of a patient's heart of an arrhythmia based on the patient-specific 3D heart model and electrocardiogram (ECG) data, the patient-specific electrical conduction map of the patient's heart identifying a localization of an initiation site of the arrhythmia;
merging the 3D localization of the initiation site of the arrhythmia and the 3D internal surface model to form an arrhythmia activation surface model;
generating a 3D model of the heart showing the wall thickness of the heart's myocardium simultaneously with the localization of an arrhythmia; and
displaying the 3D localization of the initiation site of the arrhythmia simultaneously with heart wall thickness of the 3D heart model of the myocardium for use by a physician during a cardiac electrophysiology procedure.

2. The method of claim 1, further comprising supplementing the patient-specific electrical conduction map by an internal point by point contact recording.

3. The method of claim 1, wherein generating a patient-specific 3D model of the heart including a 3D internal surface model comprises using magnetic resonance imaging (MRI) or computed tomography (CT) images of the patient to generate the patient-specific 3D heart model.

4. The method of claim 3, further comprising:
obtaining a 3D image of ECG electrodes on the patient's torso; and
merging the 3D image of the patient's torso with the 3D heart model.

5. The method of claim 4, wherein merging the 3D image of the patient's torso with the 3D patient specific heart model comprises aligning locations of ECG electrodes used in generating patient-specific electrical conduction map of a patient's heart with the ECG electrodes within the 3D image.

6. The method of claim 5, wherein ECG data obtained with 12 ECG electrodes is combined with the patient specific 3D heart model using an inverse solution calculation to generate a localization the initiation site of the arrhythmia in a heartbeat.

7. The method of claim 6, further comprising superimposing the localization point on the 3D heart model that includes myocardium wall thickness.

8. The method of claim 7, further comprising displaying heart structures including one or more of the aorta, aortic arch, pulmonary veins or coronary vessels on the displayed 3D heart model.

9. The method of claim 7, further comprising displaying heart scar tissue indicative of ischemic heart disease on the displayed 3D heart model.

10. The method of claim 7, further comprising displaying the localization of the arrhythmia as multiple points representative of multiple beats of a ventricular tachycardia on the displayed 3D heart model.

11. The method of claim 1, wherein the arrhythmia is an atrial arrhythmia.

12. The method of claim 1, wherein the arrhythmia is a ventricular arrhythmia

13. The method of claim 12, wherein the ventricular arrhythmia is a pre-ventricular contraction (PVC).

14. The method of claim 12, wherein the ventricular arrhythmia is a ventricular tachycardia.

15. The method of claim 1, wherein the arrhythmia is a dysrhythmia between the two ventricles.

16. A computing system, comprising:
a memory; and
a processor coupled to the memory and configured with processor-executable instructions to perform operations comprising:
generating a patient-specific three-dimensional (3D) heart model including a 3D internal surface model;
generating a patient-specific electrical conduction map of a patient's heart of an arrhythmia based on the patient-specific 3D heart model and electrocardiogram (ECG) data, the patient-specific electrical conduction map of the patient's heart identifying a localization of an initiation site of the arrhythmia;
merging the 3D localization of the initiation site of the arrhythmia and the 3D internal surface model to form an arrhythmia activation surface model;
generating a 3D model of the heart showing the wall thickness of the heart's myocardium simultaneously with the localization of an arrhythmia; and
displaying the 3D localization of the initiation site of the arrhythmia simultaneously with heart wall thickness of the 3D heart model of the myocardium for use by a physician during a cardiac electrophysiology procedure.

17. The computing system of claim 16, wherein the processor is configured with processor-executable instructions to perform operations further comprising supplementing the patient-specific electrical conduction map by an internal point by point contact recording.

18. The computing system of claim 16, wherein the processor is configured with processor-executable instructions to perform operations such that merging the 3D localization of the initiation site of the arrhythmia and the 3D internal surface model to form an arrhythmia activation surface model comprises using magnetic resonance imaging (MRI) or computed tomography (CT) images of the patient to generate the patient-specific 3D heart model displaying the myocardium wall thickness.

19. The computing system of claim 16, wherein the processor is configured with processor-executable instructions to perform operations further comprising:
obtaining a 3D image of ECG electrodes on the patient's torso; and
merging the 3D image of the patient's torso with the selected 3D heart model.

20. The computing system of claim 19, wherein the processor is configured with processor-executable instructions to perform operations such that merging the 3D image of the patient's torso with the 3D patient specific heart model comprises aligning locations of ECG electrodes used in generating patient-specific electrical conduction map of a patient's heart with the ECG electrodes within the 3D image.

21. The computing system of claim 20, wherein the processor is configured with processor-executable instructions to perform operations further comprising combining ECG data obtained with 12 ECG electrodes with the patient specific 3D heart model using an inverse solution calculation to generate a localization the initiation site of the arrhythmia in a heartbeat.

22. The computing system of claim 21, wherein the processor is configured with processor-executable instructions to perform operations further comprising superimposing the localization point on the 3D heart model that includes myocardium wall thickness.

23. The computing system of claim 21, wherein the processor is configured with processor-executable instructions to perform operations further comprising displaying heart structures including one or more of the aorta, aortic arch, pulmonary veins or coronary vessels on the displayed 3D heart model.

24. The computing system of claim 21, wherein the processor is configured with processor-executable instructions to perform operations further comprising displaying heart scar tissue indicative of ischemic heart disease on the displayed 3D heart model.

25. The computing system of claim 21, wherein the processor is configured with processor-executable instructions to perform operations further comprising displaying the localization as multiple points representative of multiple beats of a ventricular tachycardia on the displayed 3D heart model.
